(19) United States
(12) Patent Application Publication
(10) Pub. No.: US 2004/0258740 A1
(43) Pub. Date: Dec. 23, 2004

(54) Transdermal delivery composition

(75) Inventor: William Alejandro Thompson, Boise, ID (US)

Correspondence Address: GREENBLUM & BERNSTEIN, P.L.C., 1950 ROLAND CLARKE PLACE, RESTON, VA, 20191, US

(73) Assignee: NENE LABS, Boise, ID

(21) Appl. No.: 10/820,694

(22) Filed: Apr. 9, 2004

Related U.S. Application Data
(60) Provisional application No. 60/462,120, filed on Apr. 10, 2003.

(51) Int. Cl. A61K 31/60; A61K 31/19; A61L 15/16; A61K 31/405

(52) U.S. Cl. 424/448; 514/165; 514/420; 514/569; 514/570; 514/629

(57) ABSTRACT

A transdermal delivery composition comprising at least one skin permeation enhancer and at least one active ingredient or pharmaceutically acceptable salt thereof. The combination of components in the composition of the present invention enhances transdermal absorption of the active ingredient. This abstract is neither intended to define the invention disclosed in this specification nor intended to limit the scope of the invention in any way.

### CROSS REFERENCES TO RELATED APPLICATIONS

This application claims the benefit of U.S. Provisional Patent Application No. 60/462,120, in the names of William A. Thompson, entitled “Effervescent bath tablets containing a delivery system capable of transporting desired active ingredients transdermally,” filed on Apr. 10, 2003; the disclosure of which is expressly incorporated herein by reference in its entirety.

### BACKGROUND OF THE INVENTION

1. Field of the Invention

The present invention relates to novel pharmaceutical compositions comprising at least one skin penetration enhancer and at least one active ingredient for transdermal delivery. The composition of the present invention is unique in two respects. First, the composition of the present invention provides enhanced transdermal delivery of drugs reducing drug-related side effects associated with oral and/or parenteral administration. Second, the synergistic combination of components in the composition of the present invention increases absorption of drugs across the skin. The skin permeation enhancer adsorbs to the skin and carries the dissolved drug across the skin layers. When an effervescent agent is present, the dispersant effect of the effervescent agent increases drug contact with the skin.

Due to the combination of skin permeation enhancer and effervescent agent in one aspect of the present invention, complete drug dissolution in the enhancer portion is not required to achieve enhanced drug absorption across the skin since the effervescent portion disperses the drug to increase drug contact with the skin

Specifically, the present invention may be used for the treatment and/or alleviation of pain, aches, and inflammation, or ailments related to such symptoms.

The present invention also relates to a method of preparing pharmaceutical compositions for transdermal delivery, and methods of using the pharmaceutical compositions for the treatment and/or alleviation of pain, aches, and inflammation, and ailments associated with such symptoms.

2. Discussion of Background Information

The present invention provides a new route of delivery important for avoiding the side effects associated with oral and/or parenteral administration of anti-inflammatory drugs such as nonsteroidal anti-inflammatory drugs (NSAIDs). Transdermal delivery of drugs may also be important in reducing side effects in individuals with a reduced ability to metabolize drugs properly. An example is older patients who are more likely to be taking multiple drugs, have higher blood levels of drugs because of renal or hepatic dysfunction, and have pre-existing cognitive impairments that make it difficult to detect the role of drugs in causing new symptoms, or making old ones worse. The Medical Letter, Nov. 27, 2000, 1093: 111-112.

An example of drugs that cause undesired side effects along with their therapeutic effect is NSAIDs. In addition to their anti-pyretic effects, NSAIDs are used in treating pain and the signs and symptoms of pain, aches, and inflammation because of their analgesic and anti-inflammatory activity. It is commonly accepted that NSAIDs work by blocking the activity of cyclooxygenase (COX), also known as prostaglandin G/H synthase (PGHS), the enzyme that converts arachidonic acid into eicosanoids. Prostaglandins, especially prostaglandin E2 (PGE₂), which is the predominant eicosanoid detected in inflammation conditions, are mediators of pain, fever and other symptoms associated with inflammation. Inhibition of the biosynthesis of prostaglandins has been a therapeutic target of anti-inflammatory drug discovery. Two forms of COX were identified, a constitutive isoform (COX-1) and an inducible isoform (COX-2) of which expression is upregulated at sites of inflammation. Vane, J. R. et al., Proc. Nat'l. Acad. Sci. USA, 1994, 91: 2046. COX-1 is thought to play a physiological role and to be responsible for gastrointestinal and renal protection. COX-2 appears to play a pathological role and to be the predominant isoform present in inflammation conditions.

However, the therapeutic use of NSAIDs is limited because of drug associated side effects, such as nausea, vomiting, dyspepsia, and bleeding, as well as more severe life threatening side effects such as ulceration and renal toxicity. These side effects are due to the non-selective inhibition of both isoforms of COX by NSAIDs, providing both the desired (inhibition of COX-2) and deleterious effects (inhibition of COX-1).

It is believed that many of the side effects associated with NSAIDS may be avoided by transdermal administration of NSAIDS as opposed to the usual oral and parenteral administration routes. Orally administered NSAIDs cause local irritation by allowing a back diffusion of acid into the gastric mucosa and induce tissue damage. Parenteral administered NSAIDs can cause damage and bleeding because of inhibition of the biosynthesis of gastric prostaglandins, PGI₂ and PGE₂, that serve as cytoprotective agents in the gastric mucosa. Goodman and Gilman's The Pharmacological Basis of Therapeutics, Analgesic-antipyretic and anti-inflammatory agents and drugs employed in the treatment of gout, 9^{th} Ed. (Molinoff and Rudon, eds), 1996, page 622.

Therefore, there remains a need for formulations specifically tailored to enhance delivery of active ingredients while reducing deleterious side effects. The present pharmaceutical compositions contain a combination of skin permeation enhancer, effervescent agent, and active ingredient in ratios and amounts for effective transdermal delivery of the active ingredient.

### SUMMARY OF THE INVENTION

The present invention provides a pharmaceutical composition for transdermal delivery comprising at least one skin permeation enhancer and at least one active ingredient or pharmaceutically acceptable salt thereof, wherein the composition is for transdermal delivery of the active ingredient.

In one aspect, the skin permeation enhancer is present in an amount from about 0.1% by weight to about 20% by weight based on the total weight of the composition.

In another aspect, the active ingredient is present in an amount from about 0.1% by weight to about 30% by weight based on the total weight of the composition.

In another aspect, the skin permeation enhancer and active ingredient are present in a weight ratio from about 3.0:0.5 to about 0.5:3.0 based upon the total weight of the composition.

In another aspect, the composition of the present invention further comprises at least one effervescent agent present in an amount from about 10% to about 90% by weight based on the total weight of the composition.

In yet another aspect, the skin permeation enhancer comprises isopropyl myristate, cetyl palmitate, clarified sesame oil, borage, evening primrose oil, spirulina oil, sunflower oil, safflower oil, flaxseed oil, walnut oil, canola oil, soybean oil, and mixtures thereof.

In yet another aspect, the active ingredient comprises aspirin, sodium salicylate, choline magnesium trisalicylate, salsalate, diflunisal, salicylsalicylic acid, sulfasalazine, olsalazine, acetaminophen, indomethacin, sulindac, etodolac, tolmetin, diclofenac, ketorolac, ibuprofen, naproxen, flurbiprofen, ketoprofen, fenoprofen, indoprofen, oxaprozin, mefenamic acid, meclofenamic acid, piroxicam tenoxicam, meloxicam, lornoxicam, phenylbutazone, oxyphenthatrazone, nabumetone, diphenhydramine hydrochloride, clemastine fumarate, brompheniramine maleate, chlorpheniramine maleate, dexchlorpheniramine maleate, triprolidine hydrochloride, promethazine, hydroxyzine hydrochloride, terfenadine, astemizole, azatadine maleate, cyproheptadine hydrochloride, loratadine, carbinoxamine maleate, diphenylpyraline hydrochloride, phenindamine tartrate, tripelennamine hydrochloride, methdilazine hydrochloride, trimprazine tartrate, beclomethasone, dipropionate, triamcinolone acetonide, prednisone, Sumatriptan/Imitrex, Imigran, dihydroergotamine, ergotamine tartrate, dolasetron mesilate, dotarizine, flupirtine, tenosal, tolfenarmic acid, arotinolol, dihydroergocryptine, cyclosporin, azathioprine, methotrexate, glucocorticoids, penicillamine, hydroxychloroquine, colchicine, allopurinol, probenecid, sulfinpyrazone, ginseng, bilberry, black cohosh, cat's claw, cayenne, chamomile, chaste tree, cranberry, echinacea, eleuthero, ephedra, evening primrose oil, feverfew, flax, garlic, ginger, gingko, golden seal, green tea, hawthorn, kava kava, licorice, milk thistle, peppermint, saw palmetto, St. John's wort, methylsulfonyl methyl, DMSO, and combinations thereof

In yet another aspect, the effervescent agent comprises sodium bicarbonate, potassium bicarbonate, lithium bicarbonate, magnesium bicarbonate, calcium bicarbonate, ammonium bicarbonate, and mixtures thereof.

In still another aspect, the effervescent agent is further combined with at least one acid agent comprising citric acid, succinic acid, fumaric acid, adipic acid, malic acid, and mixtures thereof.

In still another aspect, the acid agent is present in an amount from about 5% to about 60% by weight based on the total weight of the composition.

A pharmaceutical composition of the present invention may be a tablet comprising:

at least one skin permeation enhancer in an amount from about 1% to about 5% by weight based on the total weight of the composition; and

at least one active ingredient present in an amount from about 1% by weight to about 10% by weight based on the total weight of the composition;

wherein the composition further comprises:

at least one effervescent agent present in an amount from about 30% to about 70% by weight based on the total weight of the composition;

at least one acid agent present in an amount from about 20% to about 40% by weight based on the total weight of the composition; and

wherein

the skin permeation enhancer comprises isopropyl myristate, clarified sesame oil, and mixtures thereof;

the effervescent agent is sodium bicarbonate;

the acid agent is citric acid; and

the active ingredient is ibuprofen.

A pharmaceutical composition of the present invention may comprise:

at least one skin permeation enhancer in an amount from about 1% to about 5% by weight based on the total weight of the composition; and

at least one active ingredient present in an amount from about 1% by weight to about 10% by weight based on the total weight of the composition;

wherein the composition further comprises

at least one effervescent agent present in an amount from about 30% to about 70% by weight based on the total weight of the composition;

at least one acid agent present in an amount from about 20% to about 40% by weight based on the total weight of the composition; and

wherein

the skin permeation enhancer comprises isopropyl myristate, cetyl palmitate, clarified sesame oil, borage, evening primrose oil, spirulina, sunflower, safflower oils, flaxseed, walnut, canola, soybean oil, and mixtures thereof;

the effervescent agent comprises sodium bicarbonate, potassium bicarbonate, lithium bicarbonate, magnesium bicarbonate, calcium bicarbonate, ammonium bicarbonate, and mixtures thereof;

the acid agent comprises citric acid, succinic acid, fumaric acid, adipic acid, malic acid, and mixtures thereof; and

the active ingredient comprises aspirin, sodium salicylate, choline magnesium trisalicylate, salsalate, diflunisal, salicylsalicylic acid, sulfasalazine, olsalazine, acetaminophen, indomethacin, sulindac, etodolac, tolmetin, diclofenac, ketorolac, ibuprofen, naproxen, flurbiprofen, ketoprofen, fenoprofen, indoprofen, oxaprozin, mefenamic acid, meclofenamic acid, piroxicam tenoxicam, meloxicam, lomoxicam, phenylbutazone, oxyphenthatrazone, nabumetone, diphenhydramine hydrochloride, clemastine fumarate, brompheniramine maleate, chlorpheniramine maleate, dexchlorpheniramine maleate, triprolidine hydrochloride, promethazine, hydroxyzine hydrochloride, terfenadine, astemizole, azatadine maleate, cyproheptadine hydrochloride, loratadine, carbinoxamine maleate, diphenylpyraline hydrochloride, phenindamine tartrate, tripelennamine hydrochloride, methdilazine hydrochloride, trimprazine tartrate, beclomethasone, dipropionate, triamcinolone acetonide, prednisone, Sumatriptan/Imitrex, Imigran, dihydroergotamine, ergotamine tartrate, dolasetron mesilate, dotarizine, flupirtine, tenosal, tolfenarmic acid, arotinolol, dihydroergocryptine, cyclosporin, azathioprine, methotrexate, glucocorticoids, penicillamine, hydroxychloroquine, colchicine, allopurinol, probenecid, sulfinpyrazone, ginseng, bilberry, black cohosh, cat's claw, cayenne, chamomile, chaste tree, cranberry, echinacea, eleuthero, ephedra, evening primrose oil, feverfew, flax, garlic, ginger, gingko, golden seal, green tea, hawthorn, kava kava, licorice, milk thistle, peppermint, saw palmetto, St. John's wort, methylsulfonyl methyl, DMSO, and combinations thereof.

In another aspect, a pharmaceutical composition of the present invention may be prepared comprising combining at least one skin permeation enhancer with at least one active ingredient or pharmaceutically acceptable salt thereof, at a temperature sufficient to dissolve the active ingredient without decomposing the active ingredient to obtain an enhancer mixture; and wherein preparation of the composition is accomplished at ambient temperature.

In one aspect of the method of preparing the present composition, the skin permeation enhancer is present in an amount from about 0.1% to about 20% by weight based on the total weight of the composition.

In another aspect of the method of preparing the present composition, the active ingredient is present in an amount from about 0.1% to about 30% by weight based on the total weight of the composition.

In yet another aspect of the method of preparing the present composition, the composition further comprises at least one effervescent agent present in an amount from about 10% to about 90% by weight based on the total weight of the composition.

In still another aspect of the method of preparing the present composition, the composition is prepared in a pharmaceutical formulation comprising tablet, gel, spray, and cream.

In another aspect of the present invention, the composition of the present invention may be used to treat and/or alleviate pain, aches, and inflammation comprising administering to a human being a pharmaceutical composition for transdermal delivery comprising:

at least skin permeation enhancer; and

at least one active ingredient or pharmaceutically acceptable salt thereof,

wherein the composition for transdermal delivery is administered to a human being at least once during a 24 hour period.

In one aspect of the method of treating and/or alleviating pain, aches, and inflammation, the composition is a tablet comprising:

at least one skin permeation enhancer in an amount from about 1% to about 5% by weight based on the total weight of the composition; and

at least one active ingredient present in an amount from about 1% by weight to about 10% by weight based on the total weight of the composition;

wherein the composition further comprises:

at least one effervescent agent present in an amount from about 30% to about 70% by weight based on the total weight of the composition;

at least one acid agent present in an amount from about 20% to about 40% by weight based on the total weight of the composition;

wherein

the skin permeation enhancer comprises isopropyl myristate, clarified sesame oil, and mixtures thereof;

the effervescent agent is sodium bicarbonate;

the acid agent is citric acid; and

the active ingredient is ibuprofen;

wherein the composition is a tablet; and

wherein the composition for transdermal delivery is administered to a human being at least twice during a 24 hour period.

In another aspect of the method of treating and/or alleviating pain, aches, and inflammation, the composition for transdermal delivery comprises:

at least one skin permeation enhancer in an amount from about 1% to about 5% by weight based on the total weight of the composition; and

at least one active ingredient present in an amount from about 1% by weight to about 10% by weight based on the total weight of the composition;

wherein the composition further comprises:

at least one effervescent agent present in an amount from about 30% to about 70% by weight based on the total weight of the composition;

at least one acid agent in an amount from about 20% to about 40% by weight based on the total weight of the composition; and

wherein

the skin permeation enhancer comprises isopropyl myristate, cetyl palmitate, clarified sesame oil, borage, evening primrose oil, spirulina, sunflower, safflower oils, flaxseed, walnut, canola, soybean oil, and mixtures thereof;

the effervescent agent comprises sodium bicarbonate, potassium bicarbonate, lithium bicarbonate, magnesium bicarbonate, calcium bicarbonate, ammonium bicarbonate, and mixtures thereof;

the acid agent comprises citric acid, succinic acid, fumaric acid, and mixtures thereof; and

the active ingredient comprises aspirin, sodium salicylate, choline magnesium trisalicylate, salsalate, diflunisal, salicylsalicylic acid, sulfasalazine, olsalazine, acetaminophen, indomethacin, sulindac, etodolac, tolmetin, diclofenac, ketorolac, ibuprofen, naproxen, flurbiprofen, ketoprofen, fenoprofen, indoprofen, oxaprozin, mefenamic acid, meclofenamic acid, piroxicam tenoxicam, meloxicam, lornoxicam, phenylbutazone, oxyphenthatrazone, nabumetone, diphenhydramine hydrochloride, clemastine fumarate, brompheniramine maleate, chlorpheniramine maleate, dexchlorpheniramine maleate, triprolidine hydrochloride, promethazine, hydroxyzine hydrochloride, terfenadine, astemizole, azatadine maleate, cyproheptadine hydrochloride, loratadine, carbinoxamine maleate, diphenylpyraline hydrochloride, phenindamine tartrate, tripelennamine hydrochloride, methdilazine hydrochloride, trimprazine tartrate, beclomethasone, dipropionate, triamcinolone acetonide, prednisone, Sumatriptan/Imitrex, Imigran, dihydroergotamine, ergotamine tartrate, dolasetron mesilate, dotarizine, flupirtine, tenosal, tolfenarmic acid, arotinolol, dihydroergocryptine, cyclosporin, azathioprine, methotrexate, glucocorticoids, penicillamine, hydroxychloroquine, colchicine, allopurinol, probenecid, sulfinpyrazone, ginseng, bilberry, black cohosh, cat's claw, cayenne, chamomile, chaste tree, cranberry, echinacea, eleuthero, ephedra, evening primrose oil, feverfew, flax, garlic, ginger, gingko, golden seal, green tea, hawthorn, kava kava, licorice, milk thistle, peppermint, saw palmetto, St. John's wort, methylsulfonyl methane, DMSO, and combinations thereof, and

wherein the composition for transdermal delivery is administered to a human being at least twice during a 24 hour period.

### DETAILED DESCRIPTION OF THE PRESENT INVENTION

The present invention provides a novel transdermal delivery composition comprising at least one skin permeation enhancer and at least one active ingredient permitting improved treatment and/or alleviation of pain, aches, and inflammation, and ailments associated with such symptoms. The skin permeation enhancers increase drug delivery across the skin by adsorbing to the skin and assisting in the transport of the dissolved active ingredient into the interstitial layers of the skin. When an effervescent agent is present in the composition of the present invention, the effervescent agent disperses the active ingredient to increase drug contact with the skin to further enhance drug absorption.

Furthermore, compositions of the present invention provide the benefit of enhancing drug absorption across the skin barriers, while reducing side effects related to (1) drugs and (2) individuals with hepatic and renal compromised functions. Additionally, the composition of the present invention does not dry out the skin.

In a preferred embodiment, the composition of the present invention rapidly dissolves or disintegrates when placed in water, and allows the skin permeation enhancers to adsorb to the skin to provide transdermal absorption of the active ingredient in order to treat and/or alleviate pain, aches, and inflammation. Due to the increased delivery of drug provided by the skin permeation enhancer, and drug dispersion provided by an effervescent agent, the composition of the present invention does not require complete dissolution of the active ingredient in order to attain increased drug absorption across the skin.

The composition of the present invention may be used with ailments associated with pain, aches, and/or inflammation including, but not limited to, headaches, migraines, colds, backaches, soft tissue injury, joint pain, cramps, pre-menstrual cramps, tense or sore muscles, sports injury or soreness, allergies, minor arthritis pain to severe arthritis pain (such as rheumatoid and gouty arthritis), skin autoimmune diseases (such as psoriasis, atopic dermatosis, and eczema), and inflamed skin conditions (such as sunburns, insect bites, poison ivy, and poison oak).

For instance, NSAIDs may be used in the present invention for the treatment and/or alleviation of pain, aches, and/or inflammation related to headaches, migraines, allergies, arthritis (such as rheumatoid and gouty arthritis), and skin autoimmune diseases (such as psoriasis, atopic dermatosis, and eczema). Antihistamines may be used for the treatment and/or alleviation of pain, aches, and/or inflammation related to allergies or colds. Anti-inflammatory drugs may be used in the present invention for the treatment and/or alleviation of pain, aches, and/or inflammation related to skin autoimmune diseases (such as psoriasis, atopic dermatosis, and eczema) and inflamed skin conditions (such as bums, insect bites, poison ivy, and poison oak). Immunosuppressants may be used in the present invention for the treatment and/or alleviation of pain, aches, and/or inflammation related to rheumatoid arthritis.

Pharmacologically active agents contemplated for modification in accordance with the present invention include, but are not limited to:

NSAIDs such as salicylic acid derivatives (e.g., aspirin, sodium salicylate, choline magnesium trisalicylate, salsalate, diflunisal, salicylsalicylic acid, sulfasalazine, and olsalazine), para-aminophenol derivatives (e.g., acetaminophen), indole and indene acetic acids (e.g., indomethacin, sulindac, and etodolac), heteroaryl acetic acids (e.g., tolmetin, diclofenac, and ketorolac), arylpropionic acids (such as ibuprofen, naproxen, flurbiprofen, ketoprofen, fenoprofen, indoprofen, and oxaprozin), fenamates (e.g., mefenamic acid and meclofenamic acid), oxicams (e.g., piroxicam and tenoxicam, meloxicam, lornoxicam), pyrazolidinediones (e.g., phenylbutazone and oxyphenthatrazone), and alkanones (such as nabumetone), and combinations thereof;

analgesics/antipyretics (e.g., aspirin, acetaminophen, ibuprofen, naproxen sodium, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride, meprobamate, and combinations thereof);

antihistamine/antipruritic drugs, such as ethanolamines (e.g., diphenhydramine, diphenhydramine hydrochloride, clemastine, clemastine fumarate, and the like), ethylenediamines (e.g., brompheniramine, brompheniramine maleate, chlorpheniramine, chlorpheniramine maleate, dexchlorpheniramine maleate, triprolidine, triprolidine hydrochloride, and the like), phenothiazines (e.g., promethazine), piperidines (e.g., hydroxzine, hydroxyzine hydrochloride, terfenadine, astemizole, azatadine, azatadine maleate, and the like), cyproheptadine, cyproheptadine hydrochloride, loratidine, carbinoxamine maleate, diphenylpyraline hydrochloride, phenindamine tartrate, tripelennamine hydrochloride, methdilazine hydrochloride, trimprazine tartrate, and combinations thereof;

anti-inflammatory drugs such as corticosteroids (e.g., beclomethasone, dipropionate, triarncinolone acetonide, and prednisone) and combinations thereof;

antimigraine agents, such as MK-462, 324C91, Phytomedicine, (S)-fluoxetine, calcium channel antagonists (e.g., nimodipine/Nimotop, flunarizine, dotarizine/FI-6026, iomerizine HCL/KB-2796, CPC-304, and CPC-317), .alpha.-dihydroergocryptine, 5-HT1 agonists, (e.g., Sumatriptan/Imitrex, Imigran, GR-85548, 311C, and GR-127607), 5-HT1D agonists, 5-HT1A antagonists, 5-HT1B antagonists (e.g., CP-93129), 5-HT1D antagonists (e.g., 1H-indole-5-ethanesulfonamide derivatvies and 1H-indole-5-methanesulfonamide), 5-HT1D receptor cloned (e.g., 5-HT1D agents), 2-thiophenecarboxamide, 3-piperidinamine, diclofenac potassium, dihydroergotamine (e.g., DHE 45.RTM.), ergotamine tartrate, dolasetron mesilate, dotarizine, flupirtine, histamine-H3 receptor agonist, indobufen, 1-azulenesulfonic acid derivatives, cholinesterase inhibitors, (e.g., S-9977), bradykinin antagonists, nitric oxide reductase inhibitors (e.g., BN-52296), nitric oxide receptor antagonists, substance P antagonists (e.g., Capsaicin/Nasocap), endopeptidase inhibitors (e.g., neutral endopeptidase, cloned), piperazine derivatives, neurokinin 1 antagonists, metergoline, dopamine D2 antagonist (e.g., metoclopramide+lysine acetyl), enkephalinase inhibitors (e.g., neutral endopeptidase), 5-HT2 antagonists (e.g., LY-053857), 5-HT3 antagonists (e.g., Dolasetron mesilate/MDL-73147, and 4H-carbazol-4-one derivatives), tenosal, tolfenamic acid, cyclooxygenase inhibitors (e.g., carbasalate/carbaspirin calcium, and tenosal/MR-Y134), alpha adrenoreceptor antagonists (e.g., arotinolol, and dihydroergocryptine), opioid agonists (e.g., flupirtine/D-9998), beta adrenergic antagonists (e.g., propranolol), valproate semisodium, propanolol hydrochloride, isometheptene mucate, dichloralphenazone, and combinations thereof;

immunosuppressants, such as cyclosporin, azathioprine, methotrexate, glucocorticoids, penicillamine, hydroxychloroquine, and combinations thereof;

antiarthritic agents, such as anti-CD4 monoclonal antibodies, phospholipase A1 inhibitor, loteprednol, tobramycin, combinations of loteprednol and tobramycin, salnacedin, amiprilose, anakinra, anergiX, anti-B7 antibody, anti-CD3H, anti-gp39, anti-MHC MAbs, antirheumatic peptides, anti-Tac(Fv)-PE40, AP-1 inhibitors, AR-324, purine nucleotide phosphorylase inhibitors (e.g., BCX-5), bindarit, CD2 antagonist (e.g., BTI-322), campath-1H, CD4 antagonist (e.g., CE9.1 and SB-210396), tumor necrosis factor antagonist (e.g., p80 TNFR, rhTNFbp, peptide T, CenTNF, thalidomide, CDP-571 and TBP-1), cobra venom factor, interleukin 1a agonist (e.g., cytogenin), interleukin 2 receptor antagonist (e.g., dacliximab), ICAM 1 antagonist (e.g., enlimomab), interleukin 1 beta converting enzyme inhibitors (e.g., ICE-inhibitors), interferons (e.g., thymocartin), interleukin-10, interleukin-13, interleukin 1 antagonist (e.g., SR-31747 and TJ-114), interleukin-2 antagonist (e.g., sirolimus), phospholipase C inhibitor, neurokinin 1 antagonist (e.g., L-733060), laflunimus, leflunomide, leucotriene antagonists, levamisole, LFA3TIP, macrocyclic lactone, MHC class II inhibitors, mizoribine, mycophenolate mofetil, NfkB inhibitors, oncolysin CD6, peldesine, pidotimod, PKC-RACK inhibitors, PNP inhibitors, reumacon, CD28 antagonist, roquinimex, RWJ-50271, subreum, T7 vector, tacrolimus, VLA antagonist (e.g., TBC-772), transforming growth factor beta agonist, methionine synthase inhibitors (e.g., vitamin B12 antagonist), adenosine A2 receptor agonist (e.g., YT-146), CD5 antagonist (e.g., zolimomab), 5-lipoxygenase inhibitor (e.g., zileuton, tenidap, and ABT-761), cyclooxygenase inhibitor (e.g., tenoxicam, talmetacin, piroxicam, piroxicam cinnamate, oxaprozin, NXTHIO, ML-3000, mofezolac, nabumetone, flurbiprofen, aceclofenac, diclofenac, and dexibuprofen), metalloproteinase inhibitor (e.g., XR-168, TNF convertase inhibitors, GI-155704A, AG-3340 and BB-2983), nitric oxide synthase inhbitors (i.e, ARL-16556), phospholipase A2 inhibitor (e.g., ARL-67974), selectin antagonist (e.g., CAM inhibitors), leucotriene B4 antagonist (e.g., CGS-25019C), collagenase inhibitor (e.g., GR-129574A), cyclooxygenase 2 inhibitor (e.g., meloxicam), thromboxane synthase inhibitor (e.g., curcumin), cysteine protease inhibitor (e.g., GR-373), metalloproteinase inhibitor (D-5410), lipocortins synthesis agonist (e.g., rimexolone, predonisolone 21-farnesylate, HYC-141, and deflazacort), chelating agent (diacerein), elastase inhibitors, DNA directed RNA polymerase inhibitor (e.g., estrogens), oxygen radical formation antagonist (e.g., glucosamine sulfate), thrombin inhibitors (e.g., GS-522), collagen inhibitors (e.g., halofuguinone), hyaluronic acid agonist (e.g., NRD-101, hylan, Dispasan, and Hyalart), nitric oxide antagonists (e.g., hydroxocobalamin), stromelysin inhibitors (e.g., L-758354), prostaglandin E1 agonist (e.g., misoprostol, and misoprostol+diclofenac), dihydrofolate reductase inhibitor (e.g., trimetrexate, and MX-68), opioid antagonist (e.g., nalmefene), corticotropin releasing factor antagonist (e.g., NBI-103, and NBI-104), proteolytic enzyme inhibitor (e.g., protease nexin-1, and NCY-2010), bradykinin antagonist (e.g., tachykinin antagonists, and NPC-17731), growth hormone antagonist (e.g., octreotide), phosphodiesterase IV inhibitor (e.g., PDEIV inhibitors), gelatinase inhibitor (e.g., REGA-3G12), free radical scavengers (e.g., SIDR-1026), prostaglandin synthase inhibitors (e.g., sulfasalazine), phenylbutazone, penicillamine, salsalate, azathioprine, indomethacin, meclofenamate sodium, gold sodium thiomalate, ketoprofen, auranofin, aurothioglucose, tolmetin sodium, and combinations thereof;

antigout agents (e.g., colchicine, allopurinol, probenecid, sulfinpyrazone, and combinations thereof);

antibacterial agents (e.g., amikacin sulfate, aztreonam, chloramphenicol, chloramphenicol palmitate, chloramphenicol sodium succinate, ciprofloxacin hydrochloride, clindamycin hydrochloride, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, colistin sulfate, and combinations thereof);

antifungal agents (e.g., griseofulvin, ketoconazole, and combinations thereof);

antimicrobials (e.g., cephalosporins (e.g., cefazolin sodium, cephradine, cefaclor, cephapirin sodium, ceftizoxime sodium, cefoperazone sodium, cefotetan disodium, cefutoxime azotil, cefotaxime sodium, cefadroxil monohydrate, ceftazidime, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, cefuroxime sodium, and the like), penicillins (e.g., ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G potassium, penicillin G procaine, methicillin sodium, nafcillin sodium, and the like), erythromycins (e.g., erythromycin ethylsuccinate, erythromycin, erythromycin estolate, erythromycin lactobionate, erythromycin siearate, erythromycin ethylsuccinate, and the like), tetracyclines (e.g., tetracycline hydrochloride, doxycycline hyclate, minocycline hydrochloride, and the like), and combinations thereof);

anti-infectives (e.g., miconazole, vidarabine, inosine, pranobex, vidarabine, inosine prabonex, cefpimizole sodium, fradiomycin, and combinations thereof); and

neutraceuticals (e.g., ginseng, bilberry, black cohosh, cat's claw, cayenne, chamomile, chaste tree, cranberry, echinacea, eleuthero, ephedra, evening primrose oil, feverfew, flax, garlic, ginger, gingko, golden seal, green tea, hawthorn, kava kava, licorice, milk thistle, peppermint, saw palmetto, St. John's wort, methylsulfonyl methane, DMSO, and combinations thereof).

Preferably, active ingredients that may be used in the composition of the present invention include, but are not limited to, aspirin, sodium salicylate, choline magnesium trisalicylate, salsalate, diflunisal, salicylsalicylic acid, sulfasalazine, olsalazine, acetaminophen, indomethacin, sulindac, etodolac, tolmetin, diclofenac, ketorolac, ibuprofen, naproxen, flurbiprofen, ketoprofen, fenoprofen, indoprofen, oxaprozin, mefenamic acid, meclofenamic acid, piroxicam tenoxicam, meloxicam, lornoxicam, phenylbutazone, oxyphenthatrazone, nabumetone, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, nalbuphine hydrochloride, butorphanol tartrate, butalbital, phenyltoloxamine citrate, methotrimeprazine, cinnamedrine hydrochloride, meprobamate, diphenhydramine hydrochloride, clemastine fumarate, brompheniramine maleate, chlorpheniramine maleate, dexchlorpheniramine maleate, triprolidine hydrochloride, promethazine, hydroxyzine hydrochloride, terfenadine, astemizole, azatadine maleate, cyproheptadine hydrochloride, loratidine, carbinoxamine maleate, diphenylpyraline hydrochloride, phenindamine tartrate, tripelennamine hydrochloride, methdilazine hydrochloride, trimprazine tartrate, beclomethasone, dipropionate, triamcinolone acetonide, prednisone, nimodipine/Nimotop, flunarizine, Sumatriptan/Imitrex, Imigran, dihydroergotamine, ergotamine tartrate, dolasetron mesilate, dotarizine, flupirtine, Dolasetron mesilate/MDL-73147, tenosal, tolfenarnic acid, arotinolol, dihydroergocryptine, valproate semisodium, propanolol hydrochloride, isometheptene mucate, dichloralphenazone, cyclosporin, azathioprine, methotrexate, glucocorticoids, penicillamine, hydroxychloroquine, colchicine, allopurinol, probenecid, sulfinpyrazone, amikacin sulfate, aztreonam, chloramphenicol palmitate, chloramphenicol sodium succinate, ciprofloxacin hydrochloride, clindamycin hydrochloride, clindamycin palmitate, clindamycin phosphate, metronidazole hydrochloride, gentamicin sulfate, lincomycin hydrochloride, tobramycin sulfate, vancomycin hydrochloride, polymyxin B sulfate, colistimethate sodium, colistin sulfate, griseofulvin, ketoconazole, cefazolin sodium, cephradine, cefaclor, cephapirin sodium, ceftizoxime sodium, cefoperazone sodium, cefotetan disodium, cefutoxime azotil, cefotaxime sodium, cefadroxil monohydrate, ceftazidime, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, cefuroxime sodium, ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G potassium, penicillin G procaine, methicillin sodium, nafcillin sodium, erythromycins, tetracyclines, miconazole, vidarabine, inosine, pranobex, vidarabine, inosine prabonex, cefpimizole sodium, fradiomycin, ginseng, bilberry, black cohosh, cat's claw, cayenne, chamomile, chaste tree, cranberry, echinacea, eleuthero, ephedra, evening primrose oil, feverfew, flax, garlic, ginger, gingko, golden seal, green tea, hawthorn, kava kava, licorice, milk thistle, peppermint, saw palmetto, St. John's wort, methylsulfonyl methane, DMSO, and combinations thereof.

More preferably, active ingredients that may be used in the composition of the present invention include, but are not limited to, aspirin, sodium salicylate, choline magnesium trisalicylate, salsalate, diflunisal, salicylsalicylic acid, sulfasalazine, olsalazine, acetaminophen, indomethacin, sulindac, etodolac, tolmetin, diclofenac, ketorolac, ibuprofen, naproxen, flurbiprofen, ketoprofen, fenoprofen, indoprofen, oxaprozin, mefenamic acid, meclofenamic acid, piroxicam tenoxicam, meloxicam, lornoxicam, phenylbutazone, oxyphenthatrazone, nabumetone, hydromorphone hydrochloride, oxycodone hydrochloride, codeine phosphate, dihydrocodeine bitartrate, hydrocodone bitartrate, levorphanol tartrate, butorphanol tartrate, diphenhydramine hydrochloride, clemastine fumarate, brompheniramine maleate, chlorpheniramine maleate, dexchlorpheniramine maleate, triprolidine hydrochloride, promethazine, hydroxyzine hydrochloride, terfenadine, astemizole, azatadine maleate, cyproheptadine hydrochloride, loratidine, carbinoxamine maleate, diphenylpyraline hydrochloride, phenindamine tartrate, tripelennamine hydrochloride, methdilazine hydrochloride, trimprazine tartrate, beclomethasone, dipropionate, triamcinolone acetonide, prednisone, Sumatriptan/Imitrex, Imigran, dihydroergotamine, ergotamine tartrate, dolasetron mesilate, dotarizine, flupirtine, tenosal, tolfenarmic acid, arotinolol, dihydroergocryptine, cyclosporin, azathioprine, methotrexate, glucocorticoids, penicillamine, hydroxychloroquine, colchicine, allopurinol, probenecid, sulfinpyrazone, chloramphenicol palmitate, chloramphenicol sodium succinate, ciprofloxacin hydrochloride, clindamycin hydrochloride, clindamycin palmitate, clindamycin phosphate, metronidazole hydrochloride, griseofulvin, ketoconazole, cefazolin sodium, cephradine, cefaclor, cephapirin sodium, ceftizoxime sodium, cefoperazone sodium, cefotetan disodium, cefutoxime azotil, cefotaxime sodium, cefadroxil monohydrate, ceftazidime, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, cefuroxime sodium, ampicillin, amoxicillin, erythromycins, tetracyclines, miconazole, ginseng, bilberry, black cohosh, cat's claw, cayenne, chamomile, chaste tree, cranberry, echinacea, eleuthero, ephedra, evening primrose oil, feverfew, flax, garlic, ginger, gingko, golden seal, green tea, hawthorn, kava kava, licorice, milk thistle, peppermint, saw palmetto, St. John's wort, methylsulfonyl methane, DMSO, and combinations thereof.

Most preferably, active ingredients that may be used in the composition of the present invention include, but are not limited to, aspirin, sodium salicylate, choline magnesium trisalicylate, salsalate, diflunisal, salicylsalicylic acid, sulfasalazine, olsalazine, acetaminophen, indomethacin, sulindac, etodolac, tolmetin, diclofenac, ketorolac, ibuprofen, naproxen, flurbiprofen, ketoprofen, fenoprofen, indoprofen, oxaprozin, mefenamic acid, meclofenamic acid, piroxicam tenoxicam, meloxicam, lornoxicam, phenylbutazone, oxyphenthatrazone, nabumetone, diphenhydramine hydrochloride, clemastine fumarate, brompheniramine maleate, chlorpheniramine maleate, dexchlorpheniramine maleate, triprolidine hydrochloride, promethazine, hydroxyzine hydrochloride, terfenadine, astemizole, azatadine maleate, cyproheptadine hydrochloride, loratadine, carbinoxamine maleate, diphenylpyraline hydrochloride, phenindamine tartrate, tripelennamine hydrochloride, methdilazine hydrochloride, trimprazine tartrate, beclomethasone, dipropionate, triamcinolone acetonide, prednisone, Sumatriptan/Imitrex, Imigran, dihydroergotamine, ergotamine tartrate, dolasetron mesilate, dotarizine, flupirtine, tenosal, tolfenarmic acid, arotinolol, dihydroergocryptine, cyclosporin, azathioprine, methotrexate, glucocorticoids, penicillamine, hydroxychloroquine, colchicine, allopurinol, probenecid, sulfinpyrazone, ginseng, bilberry, black cohosh, cat's claw, cayenne, chamomile, chaste tree, cranberry, echinacea, eleuthero, ephedra, evening primrose oil, feverfew, flax, garlic, ginger, gingko, golden seal, green tea, hawthorn, kava kava, licorice, milk thistle, peppermint, saw palmetto, St. John's wort, methylsulfonyl methane, DMSO, and combinations thereof.

The active ingredient is present in the composition of the present invention in an amount preferably from about 0.1% to about 30%, more preferably from about 0.5% to about 20%, and most preferably from about 1% to about 10% by weight based on the total weight of the composition.

Compositions according to the present invention comprise at least one skin permeation enhancer conventionally used in the art preferably including, but not limited to, C₁ to C₈ fatty acid esters, essential oils containing essential fatty acids such as C₁₂₋₂₄ mono- or poly-unsaturated fatty acid or alcohol (e.g. vaccenic, cis-vaccenic, linoleic, linolenic, elaidic oleic, petroselinic, erucic or nervonic acid or any of their corresponding alcohols, especially oleic acid or oleyl alcohol), C₁ to C₈ esters of C₈ to C₁₈ fatty acids, C₈ to C₁₈ fatty alcohols, sorbate esters and salts, glycerol esters of fatty acids, C₇ to C₂₂ fatty acid esters of a-hydroxy acids, and mixtures thereof.

Solvents may also be used in the present invention as a skin permeation enhancer. Such solvents include, but are not limited to, C₂ to C₇ alcohols, C₃ or C₄ diols, ethoxydiglycol, DMSO, DMF, DMA, 1-n-dodecyl-cyclazacycloheptan-2-one, N-methyl-pyrrolidone, N-(-2-hydroxyethyl)pyrrolidone, and mixtures thereof. Solvents may be present in any functional amount to facilitate dissolution of the active ingredient. Other Penetration enhancers conventionally used in the art such as those disclosed in Remington's Pharmaceutical Sciences, Eighteenth Edition, 1990 (Mack Publishing Company), are herein incorporated by reference, and may be used in the present invention.

More preferably, skin permeation enhancers that may be used in the present composition include, but are not limited to, C₁ to C₈ fatty acid esters, essential oils containing essential fatty acids such as C₁₂₋₂₄ mono- or poly-unsaturated fatty acid or alcohol (e.g. vaccenic, cis-vaccenic, linoleic, linolenic, elaidic oleic, petroselinic, erucic or nervonic acid or any of their corresponding alcohols, especially oleic acid or oleyl alcohol), C₈ to C₁₈ fatty alcohols, sorbate esters and salts, C₇ to C₂₂ fatty acid esters of α-hydroxy acids, C₂ to C₇ alcohols, C₃ or C₄ diols, DMSO, and mixtures thereof.

Most preferably, fatty acid esters, essential oils, and mixtures thereof are used as skin permeation enhancers in the present invention. The fatty acid esters that may be used in the present invention include, but are not limited to, isopropyl myristate, cetyl palmitate, and mixtures thereof. The essential oils that may be used in the present invention include, but are not limited to, clarified sesame oil, borage, evening primrose oil, spirulina, sunflower, safflower oils, flaxseed, walnut, canola, soybean oil, and mixtures thereof.

Skin permeation enhancers may be present in any functional amount and will generally be present in an amount from about 0.01% to about 30% by weight based on the total weight of the composition. Preferably, the enhancers are present in an amount from about 0.1% to about 20%, more preferably from about 0.5% to about 10%, and most preferably from about 1% to about 5% by weight based on the total weight of the composition.

In one embodiment of the present invention, where the active ingredient is at least one NSAID, the skin permeation enhancer and active ingredient are present in the composition of the present invention in a weight ratio preferably from about 3.0:0.5 to about 0.5:3.0, more preferably from about 2.5:0.75 to about 0.75:2.5, and most preferably from about 2.0:1.0 to about 1.0:2.0 based upon the total weight of the composition. These ratios may be present in any functional amount and modified in any manner known to one of skill in the art to accommodate dissolution of the active ingredient.

In another embodiment of the present invention, at least one effervescent agent may be present in the composition of the present invention. “Effervescence” as used herein includes, but is not limited to, the formation of gas, gas bubbles, foam, mousse, etc. from at least one effervescent agent as described herein.

Any suitable effervescent agent known to those skilled in the art can be used in the present invention so long as the effervescent agent's pH, when physically separated from the acid agent, is about 7 or more, preferably from about 7 to about 11, more preferably from about 8 to about 9.

Suitable bases for use as the effervescent agent include, but are not limited to, carbonates, bicarbonates, sesquicarbonates and mixtures thereof. Preferably, the base is selected from the group consisting of sodium carbonate, potassium carbonate, lithium carbonate, magnesium carbonate, calcium carbonate, ammonium carbonate, mono-, di-, tri- or tetra-alkyl or aryl, substituted or unsubstituted, ammonium carbonate, sodium bicarbonate, potassium bicarbonate, lithium bicarbonate, magnesium bicarbonate, calcium bicarbonate, ammonium bicarbonate, mono-, di-, tri- or tetra-alkyl or aryl, substituted or unsubstituted, ammonium bicarbonate, and mixtures thereof. More preferably, the base is selected from the group consisting of sodium carbonate, magnesium carbonate, calcium carbonate, sodium bicarbonate, potassium bicarbonate, lithium bicarbonate, magnesium bicarbonate, calcium bicarbonate, ammonium bicarbonate, and mixtures thereof. The most preferred bases are selected from the group consisting of sodium bicarbonate, potassium bicarbonate, lithium bicarbonate, magnesium bicarbonate, calcium bicarbonate, ammonium bicarbonate, and mixtures thereof.

The effervescent agent preferably comprises a base, preferably present at a level of from about 10% to about 90%, more preferably from about 20% to about 80%, and most preferably from about 40% to about 60% by weight based on the total weight of the composition of the present invention.

The effervescent agent used in the present invention may also be used with any suitable acid known to those skilled in the art so long as the acid agent's pH, when physically separated from the effervescent agent, is about 7 or less, preferably from about 0 to about 6, more preferably from about 3 to about 4.

Suitable acids for use with the effervescent agent include acids that have a pKa of 7 or less, preferably from about 3 to about 7.

Non-limiting examples of suitable acids for use in the present invention include inorganic acids, organic acids, and mixtures thereof. Preferably, the inorganic acids are selected from the group consisting of sulfuric acid, hydrochloric acid, phosphoric acid, nitric acid, and mixtures thereof. Preferably, the organic acids are selected from the group consisting of formic acid, acetic acid, C.sub.12-C.sub.18 fatty acids, malic acid, maleic acid, malonic acid, succinic acid, tartaric acid, lactic acid, glutaric acid, fumaric acid, benzoic acid, phthalic acid, citric acid, adipic acid, and mixtures thereof. More preferably, the organic acids are malic acid, maleic acid, succinic acid, tartaric acid, lactic acid, fumaric acid, benzoic acid, phthalic acid, citric acid, adipic acid, and mixtures thereof. Most preferably, the organic acids are citric acid, succinic acid, fumaric acid, adipic acid, malic acid, and mixtures thereof.

Preferably, the acid agent is present at an amount from about 5% to about 60%, more preferably from about 10% to about 50%, and most preferably from about 20% to about 40% by weight based on the total weight of the composition of the present invention.

The topical formulation of the present invention may contain pharmaceutically acceptable excipients conventionally known and used in the art including, but not limited to, surfactants, disintegrants, binders, lubricants, wetting agents, dispersing agents, emulsifiers, penetrants, emollients, detergents, hardeners, fillers, antioxidants (e.g., fumaric acid, malic acid, propyl gallate, ascorbic acid (vitamin C), vitamin A (retinal), ascorbyl palmirate, N-acetylcsysteine, butyl hydroxy anisole (BHA), butyl hydroxy toluene (BHT), β-Carotene, flavonoids, glutathione, α-lipoic acid, melatonin, and tocopherols, e.g. .alpha.-tocopherol (vitamin E), cooling agents such as menthol, sooth agents such as camphor, aloe, and panthenol, or coloring agents such as zinc oxide.

Additives such as thickening agents, gums, preservatives, fragrances, stabilizers, antioxidants, agents to increase the solubility of the active ingredients, and the like can also be incorporated into the formulation. Examples of such additives include, but are not limited to butylated hydroxytoluene.

Examples of disintegrating agents include, for example, celluloses such as sodium croscarmellose, calcium carmelose, carmelose, low-substituted hydroxypropyl cellulose, etc.; starches such as sodium carboxymethyl starch, hydroxypropyl starch, partly pregelatinized starch, etc.; and crospovidone.

Examples of binders include, for example, celluloses such as hydroxypropyl cellulose, hydroxypropylmethyl cellulose, methyl cellulose, etc.; starches such as pregelatinized starch, starch paste, etc.; synthetic polymers such as poly(vinylpyrrolidone)s, carboxyvinyl polymers, etc.; and natural polymers such as sodium alginate, xanthan gum, gum arabic, etc.

Examples of lubricants include magnesium stearate, calcium stearate, stearic acid, sucrose fatty acid esters, talc, etc.

In a preferred embodiment of the present invention, the composition of the present invention is prepared at ambient temperature (e.g., 25° C.), using Good Manufacturing Practices (GMPs) standards. The composition of the present invention is prepared in a process that includes combining at least one skin permeation enhancer in an enhancer mixture with at least one active ingredient or pharmaceutically acceptable salt thereof. To aid in dissolution of the active ingredient, the enhancer mixture is heated to any temperature to accommodate dissolution of the active ingredient so long as the temperature does not cause decomposition of the active ingredient. The dissolution of the active ingredient in the present invention may be controlled by modification of the skin permeation enhancer.

More preferably, the composition of the present invention is prepared by blending two effervescent agents until well mixed to make up the effervescent mixture. The effervescence allows effective dispersion of the composition of the present invention in water. Two skin permeation enhancers are then blended until homogeneous to make up the enhancer mixture. The enhancer mixture is heated to a temperature of about 40° C. An active ingredient is then added to the enhancer mixture and mixed until desired dissolution of the active ingredient is achieved. The enhancer mixture is combined with the effervescent mixture until well blended, and the final mixture possesses a flaky consistency. The final flaky mixture is pressed into tablet molds. The tablets are pressed using a tablet press according to methods known in the art, and allowed to dry for a few hours. The result is an effervescent tablet which when dissolved in water (such as bathtub water), allows the skin permeation enhancers to adsorb to the skin and allow transdermal delivery of the active ingredient.

Most preferably, the composition of the present invention is prepared by mixing sodium bicarbonate and citric acid together as dry ingredients to form the effervescent mixture. dl-Panthenol and butylated hydroxytoluene may be added in discretionary amounts to the effervescent mixture and adjusted without adversely affecting the transdermal absorption of the active ingredients. Isopropyl myristate and clarified sesame oil are then mixed together to obtain a clear, homogeneous enhancer mixture. The enhancer mixture is heated to 40° C., and ibuprofen is added and blended. The active ingredient will not completely dissolve. The drug mixture is then blended with the dry effervescent mixture, and divided into 50 gram batches each of which may be pressed into tablet molds and allowed to dry for a few hours. The result is an effervescent tablet which dissolves when placed in water.

The molded tablets of the present invention is useful as a rapidly soluble molded tablet because it is rapidly disintegrated and dissolved immediately after being put in water, and it has a proper hardness. Usually, the hardness of the molded tablet is preferably about 1 to about 20 kg. The hardness of the molded tablet is preferably, in particular, about 2 to about 12 kg, more preferably about 5 to about 10 kg.

The rapidly disintegrable tablet of the present invention can be produced according to a conventional process for producing a molded tablet.

The tablet molds range in size from 10 gram to 400 gram tablet size. The tablet size of the present invention are preferably 20 gram to 400 gram tablets for use in an industry standard sized bathtub. More preferably, the tablet size of the present invention are 30 gram to 350 gram tablets for use in an industry standard sized bathtub. Most preferably, the tablet size of the present invention are 50 gram to 300 gram tablets for use in an industry standard sized bathtub.

Preferably, the composition of the present invention may be of varying sizes for different applications. For instance, the composition of the present invention, without limitation, may be used by dissolving one 50 gram tablet in warm water used to fill an industry standard sized bathtub allowing an individual to soak in the water. A foot bath, hand bath, or other smaller bath may require a smaller tablet to dissolve in the water.

Most importantly, the composition of the present invention may be modified to accommodate drug delivery of different active ingredients. For example, the isopropyl myristate and clarified sesame oil may be modified to increase the dissolution of the active ingredient in the composition, or modified to decrease the dissolution of the active ingredient in the composition. Complete dissolution of the active ingredient is not required to achieve effective drug absorption across the skin for with the composition of the present invention. The skin permeation enhancers such as isopropyl myristate and clarified sesame oil, may be substituted with other skin permeation enhancers disclosed above without decreasing transdermal drug absorption.

The composition of the present invention may also be formulated into other topical formulations, including but not limited to, gel, spray, and cream. Methods of preparing compositions of the present invention into topical formulations may be done using methods known to those of skill in the art. For example, conventional methods such as those disclosed in Remington's Pharmaceutical Sciences, 18^{th} Ed. may be used in the present invention for preparing gels, sprays, and creams. Additionally, the compositions of the present invention have a stable shelf life of at least 24 months.

In a preferred embodiment of the present invention, the composition of the present invention may be administered preferably, one to six times during a 24 hour period. More preferably, the composition of the present invention may be administered at least once during a 24 hour period. Most preferably, the composition of the present invention may be administered at least twice during a 24 hour period of time. Fractional or other doses may be taken simultaneously or at different times during the 24 hour period. When employing the compositions of the present invention, pain relief may be noted in about 15 to 20 minutes.

The composition of the present invention, without limitation, may be used alone or in combination with a therapeutic therapy or regimen. The therapeutic therapy or regimen may be for treating symptoms associated with pain, aches, and/or inflammation, or may be entirely unrelated to such symptoms. For example, the present method may be incorporated as part of arthritis or allergy therapy, or in combination with cancer therapy.

In a most preferred embodiment of the present invention, the composition of the present invention comprises:

at least one skin permeation enhancer in an amount from about 1% to about 5% by weight based on the total weight of the composition; and

at least one active ingredient present in an amount from about 1% by weight to about 10% by weight based on the total weight of the composition;

wherein the composition further comprises:

at least one effervescent agent present in an amount from about 30% to about 70% by weight based on the total weight of the composition; and

at least one acid agent in an amount from about 20% to about 40% by weight based on the total weight of the composition; and

wherein

the skin permeation enhancer comprises isopropyl myristate, cetyl palmitate, clarified sesame oil, borage, evening primrose oil, spirulina, sunflower, safflower oils, flaxseed, walnut, canola, soybean oil, and mixtures thereof;

the effervescent agent comprises sodium bicarbonate, potassium bicarbonate, lithium bicarbonate, magnesium bicarbonate, calcium bicarbonate, ammonium bicarbonate, and mixtures thereof;

the acid agent comprises citric acid, succinic acid, fumaric acid, and mixtures thereof; and

the active ingredient comprises aspirin, sodium salicylate, choline magnesium trisalicylate, salsalate, diflunisal, salicylsalicylic acid, sulfasalazine, olsalazine, acetaminophen, indomethacin, sulindac, etodolac, tolmetin, diclofenac, ketorolac, ibuprofen, naproxen, flurbiprofen, ketoprofen, fenoprofen, indoprofen, oxaprozin, mefenamic acid, meclofenamic acid, piroxicam tenoxicam, meloxicam, lornoxicam, phenylbutazone, oxyphenthatrazone, nabumetone, diphenhydramine hydrochloride, clemastine fumarate, brompheniramine maleate, chlorpheniramine maleate, dexchlorpheniramine maleate, triprolidine hydrochloride, promethazine, hydroxyzine hydrochloride, terfenadine, astemizole, azatadine maleate, cyproheptadine hydrochloride, loratadine, carbinoxamine maleate, diphenylpyraline hydrochloride, phenindamine tartrate, tripelennamine hydrochloride, methdilazine hydrochloride, trimprazine tartrate, beclomethasone, dipropionate, triamcinolone acetonide, prednisone, Sumatriptan/Imitrex, Imigran, dihydroergotamine, ergotamine tartrate, dolasetron mesilate, dotarizine, flupirtine, tenosal, tolfenarmic acid, arotinolol, dihydroergocryptine, cyclosporin, azathioprine, methotrexate, glucocorticoids, penicillamine, hydroxychloroquine, colchicine, allopurinol, probenecid, sulfinpyrazone, ginseng, bilberry, black cohosh, cat's claw, cayenne, chamomile, chaste tree, cranberry, echinacea, eleuthero, ephedra, evening primrose oil, feverfew, flax, garlic, ginger, gingko, golden seal, green tea, hawthorn, kava kava, licorice, milk thistle, peppermint, saw palmetto, St. John's wort, methylsulfonyl methyl, DMSO, and combinations thereof.

In still another most preferred embodiment of the present invention, the composition of the present invention is a tablet comprising:

at least one skin permeation enhancer in an amount from about 1% to about 5% by weight based on the total weight of the composition; and

at least one active ingredient present in an amount from about 1% by weight to about 10% by weight based on the total weight of the composition;

wherein the composition further comprises:

at least one effervescent agent present in an amount from about 30% to about 70% by weight based on the total weight of the composition;

at least one acid agent present in an amount from about 20% to about 40% by weight based on the total weight of the composition; and

wherein

the skin permeation enhancer comprises isopropyl myristate, clarified sesame oil, and mixtures thereof;

the effervescent agent is sodium bicarbonate;

the acid agent is citric acid; and

the active ingredient is ibuprofen.

In another most preferred embodiment of the present invention, a method of treating and/or alleviating pain, aches, and inflammation is contemplated comprising administering to a human being a pharmaceutical composition of the present invention comprising:

at least one skin permeation enhancer in an amount from about 1% to about 5% by weight based on the total weight of the composition; and

at least one active ingredient present in an amount from about 1% by weight to about 10% by weight based on the total weight of the composition;

wherein the composition further comprises

at least one effervescent agent present in an amount from about 30% to about 70% by weight based on the total weight of the composition;

at least one acid agent in an amount from about 20% to about 40% by weight based on the total weight of the composition;

wherein

the skin permeation enhancer comprises isopropyl myristate, cetyl palmitate, clarified sesame oil, borage, evening primrose oil, spirulina, sunflower, safflower oils, flaxseed, walnut, canola, soybean oil, and mixtures thereof;

the effervescent agent comprises sodium bicarbonate, potassium bicarbonate, lithium bicarbonate, magnesium bicarbonate, calcium bicarbonate, ammonium bicarbonate, and mixtures thereof;

the acid agent comprises citric acid, succinic acid, fumaric acid, and mixtures thereof; and

the active ingredient comprises aspirin, sodium salicylate, choline magnesium trisalicylate, salsalate, diflunisal, salicylsalicylic acid, sulfasalazine, olsalazine, acetaminophen, indomethacin, sulindac, etodolac, tolmetin, diclofenac, ketorolac, ibuprofen, naproxen, flurbiprofen, ketoprofen, fenoprofen, indoprofen, oxaprozin, mefenamic acid, meclofenamic acid, piroxicam tenoxicam, meloxicam, lornoxicam, phenylbutazone, oxyphenthatrazone, nabumetone, diphenhydramine hydrochloride, clemastine fumarate, brompheniramine maleate, chlorpheniramine maleate, dexchlorpheniramine maleate, triprolidine hydrochloride, promethazine, hydroxyzine hydrochloride, terfenadine, astemizole, azatadine maleate, cyproheptadine hydrochloride, loratadine, carbinoxamine maleate, diphenylpyraline hydrochloride, phenindamine tartrate, tripelennamine hydrochloride, methdilazine hydrochloride, trimprazine tartrate, beclomethasone, dipropionate, triamcinolone acetonide, prednisone, Sumatriptan/Imitrex, Imigran, dihydroergotamine, ergotamine tartrate, dolasetron mesilate, dotarizine, flupirtine, tenosal, tolfenarmic acid, arotinolol, dihydroergocryptine, cyclosporin, azathioprine, methotrexate, glucocorticoids, penicillamine, hydroxychloroquine, colchicine, allopurinol, probenecid, sulfinpyrazone, ginseng, bilberry, black cohosh, cat's claw, cayenne,.chamomile, chaste tree, cranberry, echinacea, eleuthero, ephedra, evening primrose oil, feverfew, flax, garlic, ginger, gingko, golden seal, green tea, hawthorn, kava kava, licorice, milk thistle, peppermint, saw palmetto, St. John's wort, methylsulfonyl methyl, DMSO, and combinations thereof; and

wherein the composition for transdermal delivery is administered to a human being at least twice during a 24 hour period.

In still another most preferred embodiment of the present invention, a method of treating and/or alleviating pain, aches, and inflammation is contemplated comprising administering to a human being a pharmaceutical composition of the present invention comprising:

at least one skin permeation enhancer in an amount from about 1% to about 5% by weight based on the total weight of the composition; and

at least one active ingredient present in an amount from about 1% by weight to about 10% by weight based on the total weight of the composition;

wherein the composition further comprises:

at least one effervescent agent present in an amount from about 30% to about 70% by weight based on the total weight of the composition;

at least one acid agent present in an amount from about 20% to about 40% by weight based on the total weight of the composition;

wherein

the skin permeation enhancer comprises isopropyl myristate, clarified sesame oil and mixtures thereof;

the effervescent agent is sodium bicarbonate;

the acid agent is citric acid;

the active ingredient is ibuprofen;

wherein the composition is a tablet; and

wherein the composition for transdermal delivery is administered to a human being at least twice during a 24 hour period.

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description making apparent to those skilled in the art how the several forms of the present invention may be embodied in practice.

### EXAMPLE 1

### Pharmaceutical Formulations

The following are illustrative pharmaceutical formulations which may be employed in practicing the present invention:

A. Tablet dosage form:

| | | |
|---|---|---|
| Isopropyl myristate | 1% to | 3% by weight |
| Clarified sesame oil | 1% to | 3% by weight |
| Citric acid | 20% to | 40% by weight |
| Active ingredient | 1% to | 3% by weight |
| Sodium bicarbonate | 40% to | 60% by weight |

The active ingredient may be at least one, or combination of at least one NSAID, antihistamine, anti-inflammatory drug, and immunosuppressive drug.

B. Tablet dosage form:

| | | |
|---|---|---|
| Isopropyl myristate | 1% to | 3% by weight |
| Clarified sesame oil | 1% to | 3% by weight |
| Citric acid | 20% to | 40% by weight |
| Active ingredient | 1% to | 3% by weight |
| Sodium bicarbonate | 40% to | 65% by weight |

Tablet is pressed into a 50 gram tablet.

C. Tablet dosage form:

| | |
|---|---|
| Isopropyl myristate | 1.8% by weight |
| Clarified sesame oil | 1.6% by weight |
| Citric acid | 35% by weight |
| Ibuprofen | 1.6% by weight |
| Sodium bicarbonate | 60% by weight |

Tablet is pressed into a 50 gram tablet.

D. Tablet dosage form:

| | |
|---|---|
| Isopropyl myristate | 1.8% by weight |
| Clarified sesame oil | 1.6% by weight |
| Citric acid | 35% by weight |
| Ibuprofen | 1.6% by weight |
| Ketoprofen | 1.6% by weight |
| Sodium bicarbonate | 60% by weight |

Tablet is pressed into a 50 gram tablet.

E. Tablet dosage form:

| | |
|---|---|
| Isopropyl myristate | 1.6% by weight |
| Clarified sesame oil | 1.8% by weight |
| Citric acid | 36% by weight |
| Ibuprofen | 1.6% by weight |
| Sodium bicarbonate | 59% by weight |

Tablet is pressed into a 50 gram tablet.

F. Tablet dosage form:

| | |
|---|---|
| Isopropyl myristate | 1.8% by weight |
| Clarified sesame oil | 1.6% by weight |
| Citric acid | 30% by weight |
| Ibuprofen | 1.6% by weight |
| Sodium bicarbonate | 63% by weight |
| dl-Panthenol | 1% by weight |
| Butylated hydroxytoluene | 1% by weight |

Tablet is pressed into a 50 gram tablet. dl-Panthenol and butylated hydroxytoluene may be increased or decreased without adversely affecting the transdermal absorption of the active ingredient.

### EXAMPLE 2

### Use of Effervescent Tablet Formulation for Treatment of Pain

The effervescent tablet formulation of Example 1C allows transdermal pain relief after subject has applied the composition to bath water and soaked the affected area. The analgesic effect of the active ingredient is without side effects such as stomach irritation commonly associated with drugs such as NSAIDs.

### EXAMPLE 3

### Use of Effervescent Tablet Formulation for Treatment of Pain

The effervescent tablet formulation of Example 1E (hereinafter “test composition”) was tested on 50 human subjects. Each of the subjects tested indicated that the test composition was quickly absorbed into the skin, allowing the analgesic to quickly and efficiently alleviate the area of pain. Furthermore, the test composition was not found to be irritating or dehydrating to the skin.

### EXAMPLE 4

### Use of Effervescent Tablet Formulation for Treatment of Arthritis Pain

The test composition of Example 1E was tested on arthritic fingers of a male subject. Fifteen minutes after application, there was a noted reduction in arthritis pain lasting up to 5 hours.

### EXAMPLE 5

### Method of Preparing Effervescent Tablets

A. A tablet dosage form in accordance with the present invention may be prepared in the following manner.

Step 1:

| | |
|---|---|
| (a) Citric acid | 20 to 40% by weight |
| (b) Sodium bicarbonate | 40 to 60% by weight |

(a) and (b) are blended to obtain an effervescent mixture.

Step 2:

| | |
|---|---|
| (c) Isopropyl myristate | 1 to 3% by weight |
| (d) Clarified sesame oil | 1 to 3% by weight |

(c) and (d) are blended to obtain a clear, homogeneous enhancer mixture. The enhancer mixture is heated to at temperature of about 40° C.

Step 3:

Ibuprofen is then added to the enhancer mixture. The ibuprofen does not have to be completely dissolved. The effervescent mixture is then added to the enhancer mixture until well blended, and the final mixture possesses a flaky consistency. The final flaky mixture is divided into 50 gram batches, each of which is pressed into tablet molds and allowed to dry for a few hours. The flaky mixture may also be pressed into molds of 10 grams to 100 grams in size. The result is an effervescent tablet which dissolves when placed in warm water.

B. The effervescent tablet of Example 5A may also be pressed into tablet molds of 30 grams to 90 grams in size.

### EXAMPLE 6

### Topical Application

A 50 gram effervescent tablet containing ibuprofen as the active ingredient is dissolved in warm water used to fill an industry-standard sized bath tub. A person then soaks in the tub for at least 5 minutes, or as long as desired. After soaking, the person does not wash the skin so that the ibuprofen continues to be absorbed through the skin due to the continued transdermal delivery from the presence of the skin permeation enhancer (preferably, isopropyl myristate) and effervescent agent (preferably, clarified sesame oil).

It is noted that the foregoing examples have been provided merely for the purpose of explanation and are in no way to be construed as limiting of the present invention. While the present invention has been described with reference to exemplary embodiments, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A pharmaceutical composition for transdermal delivery comprising:
at least one skin permeation enhancer; and
at least one active ingredient or pharmaceutically acceptable salt thereof,
wherein the composition is for transdermal delivery of the active ingredient.

2. The composition of claim 1 wherein the skin permeation enhancer is present in an amount from about 0.1% by weight to about 20% by weight based on the total weight of the composition.

3. The composition of claim 2 wherein the active ingredient is present in an amount from about 0.1% by weight to about 30% by weight based on the total weight of the composition.

4. The composition of claim 3 wherein the skin permeation enhancer and active ingredient are present in a weight ratio from about 3.0:0.5 to about 0.5:3.0 based upon the total weight of the composition.

5. The composition of claim 4 further comprising at least one effervescent agent wherein the effervescent agent is present in an amount from about 10% to about 90% by weight based on the total weight of the composition.

6. The composition of claim 2 wherein the skin permeation enhancer comprises isopropyl myristate, cetyl palmitate, clarified sesame oil, borage, evening primrose oil, spirulina oil, sunflower oil, safflower oil, flaxseed oil, walnut oil, canola oil, soybean oil, and mixtures thereof.

7. The composition of claim 6 wherein the active ingredient comprises aspirin, sodium salicylate, choline magnesium trisalicylate, salsalate, diflunisal, salicylsalicylic acid, sulfasalazine, olsalazine, acetaminophen, indomethacin, sulindac, etodolac, tolmetin, diclofenac, ketorolac, ibuprofen, naproxen, flurbiprofen, ketoprofen, fenoprofen, indoprofen, oxaprozin, mefenamic acid, meclofenamic acid, piroxicam tenoxicam, meloxicam, lornoxicam, phenylbutazone, oxyphenthatrazone, nabumetone, diphenhydramine hydrochloride, clemastine fumarate, brompheniramine maleate, chlorpheniramine maleate, dexchlorpheniramine maleate, triprolidine hydrochloride, promethazine, hydroxyzine hydrochloride, terfenadine, astemizole, azatadine maleate, cyproheptadine hydrochloride, loratadine, carbinoxamine maleate, diphenylpyraline hydrochloride, phenindamine tartrate, tripelennamine hydrochloride, methdilazine hydrochloride, trimprazine tartrate, beclomethasone, dipropionate, triamcinolone acetonide, prednisone, Sumatriptan/Imitrex, Imigran, dihydroergotamine, ergotamine tartrate, dolasetron mesilate, dotarizine, flupirtine, tenosal, tolfenarmic acid, arotinolol, dihydroergocryptine, cyclosporin, azathioprine, methotrexate, glucocorticoids, penicillamine, hydroxychloroquine, colchicine, allopurinol, probenecid, sulfinpyrazone, ginseng, bilberry, black cohosh, cat's claw, cayenne, chamomile, chaste tree, cranberry, echinacea, eleuthero, ephedra, evening primrose oil, feverfew, flax, garlic, ginger, gingko, golden seal, green tea, hawthorn, kava kava, licorice, milk thistle, peppermint, saw palmetto, St. John's wort, methylsulfonyl methane, DMSO, and combinations thereof.

8. The composition of claim 5 wherein the effervescent agent comprises sodium bicarbonate, potassium bicarbonate, lithium bicarbonate, magnesium bicarbonate, calcium bicarbonate, ammonium bicarbonate, and mixtures thereof.

9. The composition of claim 8 wherein the effervescent agent is further combined with at least one acid agent comprising citric acid, succinic acid, fumaric acid, adipic acid, malic acid, and mixtures thereof.

10. The composition of claim 9 wherein the acid agent is present in an amount from about 5% to about 60% by weight based on the total weight of the composition.

11. The composition of claim 10 wherein the composition is a tablet comprising:
at least one skin permeation enhancer in an amount from about 1% to about 5% by weight based on the total weight of the composition; and
at least one active ingredient present in an amount from about 1% by weight to about 10% by weight based on the total weight of the composition;
wherein the composition further comprises
at least one effervescent agent present in an amount from about 30% to about 70% by weight based on the total weight of the composition; and
at least one acid agent present in an amount from about 20% to about 40% by weight based on the total weight of the composition; and
wherein
the skin permeation enhancer comprises isopropyl myristate, clarified sesame oil, and mixtures thereof,
the effervescent agent is sodium bicarbonate;
the acid agent is citric acid; and
the active ingredient is ibuprofen.

12. The composition of claim 1, wherein the composition comprises:
at least one skin permeation enhancer in an amount from about 1% to about 5% by weight based on the total weight of the composition; and
at least one active ingredient present in an amount from about 1% by weight to about 10% by weight based on the total weight of the composition;
wherein the composition further comprises:
at least one effervescent agent present in an amount from about 30% to about 70% by weight based on the total weight of the composition;
at least one acid agent in an amount from about 20% to about 40% by weight based on the total weight of the composition; and
wherein
the skin permeation enhancer comprises isopropyl myristate, cetyl palmitate, clarified sesame oil, borage, evening primrose oil, spirulina oil, sunflower oil, safflower oil, flaxseed oil, walnut oil, canola oil, soybean oil, and mixtures thereof;
the effervescent agent comprises sodium bicarbonate, potassium bicarbonate, lithium bicarbonate, magnesium bicarbonate, calcium bicarbonate, ammonium bicarbonate, and mixtures thereof;
the acid agent comprises citric acid, succinic acid, fumaric acid, adipic acid, malic acid, and mixtures thereof; and
the active ingredient comprises aspirin, sodium salicylate, choline magnesium trisalicylate, salsalate, diflunisal, salicylsalicylic acid, sulfasalazine, olsalazine, acetaminophen, indomethacin, sulindac, etodolac, tolmetin, diclofenac, ketorolac, ibuprofen, naproxen, flurbiprofen, ketoprofen, fenoprofen, indoprofen, oxaprozin, mefenamic acid, meclofenamic acid, piroxicam tenoxicam, meloxicam, lornoxicam, phenylbutazone, oxyphenthatrazone, nabumetone, diphenhydramine hydrochloride, clemastine fumarate, brompheniramine maleate, chlorpheniramine maleate, dexchlorpheniramine maleate, triprolidine hydrochloride, promethazine, hydroxyzine hydrochloride, terfenadine, astemizole, azatadine maleate, cyproheptadine hydrochloride, loratadine, carbinoxamine maleate, diphenylpyraline hydrochloride, phenindamine tartrate, tripelennamine hydrochloride, methdilazine hydrochloride, trimprazine tartrate, beclomethasone, dipropionate, triamcinolone acetonide, prednisone, Sumatriptan/Imitrex, Imigran, dihydroergotamine, ergotamine tartrate, dolasetron mesilate, dotarizine, flupirtine, tenosal, tolfenarmic acid, arotinolol, dihydroergocryptine, cyclosporin, azathioprine, methotrexate, glucocorticoids, penicillamine, hydroxychloroquine, colchicine, allopurinol, probenecid, sulfinpyrazone, ginseng, bilberry, black cohosh, cat's claw, cayenne, chamomile, chaste tree, cranberry, echinacea, eleuthero, ephedra, evening primrose oil, feverfew, flax, garlic, ginger, gingko, golden seal, green tea, hawthorn, kava kava, licorice, milk thistle, peppermint, saw palmetto, St. John's wort, methylsulfonyl methane, DMSO, and combinations thereof.

13. A method of preparing a pharmaceutical composition for transdermal delivery comprising combining at least one skin permeation enhancer with at least one active ingredient or pharmaceutically acceptable salt thereof, at a temperature sufficient to dissolve the active ingredient without decomposing the active ingredient to obtain an enhancer mixture; and wherein preparation of the composition is accomplished at ambient temperature.

14. A method of claim 13, wherein the skin permeation enhancer is present in an amount from about 0.1% to about 20% by weight based on the total weight of the composition.

15. A method of 14, wherein the active ingredient is present in an amount from about 0.1% to about 30% by weight based on the total weight of the composition.

16. A method of claim 15, further comprising at least one effervescent agent wherein the effervescent agent is present in an amount from about 10% to about 90% by weight based on the total weight of the composition.

17. The method of claim 16, wherein the composition is prepared in a pharmaceutical formulation comprising tablet, gel, spray, and cream.

18. A method of treating and/or alleviating pain, aches, and inflammation comprising administering to a human being a pharmaceutical composition for transdermal delivery comprising:
at least skin permeation enhancer; and
at least one active ingredient or pharmaceutically acceptable salt thereof,
wherein the composition for transdermal delivery is administered to a human being at least once during a 24 hour period.

19. The method of claim 18, wherein the composition is tablet comprising:
at least one skin permeation enhancer in an amount from about 1% to about 5% by weight based on the total weight of the composition; and
at least one active ingredient present in an amount from about 1% by weight to about 10% by weight based on the total weight of the composition;
wherein the composition further comprises:
at least one effervescent agent present in an amount from about 30% to about 70% by weight based on the total weight of the composition; and
at least one acid agent present in an amount from about 20% to about 40% by weight based on the total weight of the composition;
wherein
the skin permeation enhancer comprises isopropyl myristate, clarified sesame oil and mixtures thereof;
the effervescent agent is sodium bicarbonate;
the acid agent is citric acid; and
the active ingredient is ibuprofen;
wherein the composition is a tablet; and
wherein the composition for transdermal delivery is administered to a human being at least twice during a 24 hour period.

20. The method of claim 18, wherein the composition for transdermal delivery comprises:
at least one skin permeation enhancer in an amount from about 1% to about 5% by weight based on the total weight of the composition; and
at least one active ingredient present in an amount from about 1% by weight to about 10% by weight based on the total weight of the composition;
wherein the composition further comprises
at least one effervescent agent present in an amount from about 30% to about 70% by weight based on the total weight of the composition;
at least one acid agent in an amount from about 20% to about 40% by weight based on the total weight of the composition; and
wherein
the skin permeation enhancer comprises isopropyl myristate, cetyl palmitate, clarified sesame oil, borage, evening primrose oil, spirulina oil, sunflower oil, safflower oil, flaxseed oil, walnut oil, canola oil, soybean oil, and mixtures thereof;
the effervescent agent comprises sodium bicarbonate, potassium bicarbonate, lithium bicarbonate, magnesium bicarbonate, calcium bicarbonate, ammonium bicarbonate, and mixtures thereof;
the acid agent comprises citric acid, succinic acid, fumaric acid, adipic acid, malic acid, and mixtures thereof; and
the active ingredient comprises aspirin, sodium salicylate, choline magnesium trisalicylate, salsalate, diflunisal, salicylsalicylic acid, sulfasalazine, olsalazine, acetaminophen, indomethacin, sulindac, etodolac, tolmetin, diclofenac, ketorolac, ibuprofen, naproxen, flurbiprofen, ketoprofen, fenoprofen, indoprofen, oxaprozin, mefenamic acid, meclofenamic acid, piroxicam tenoxicam, meloxicam, lornoxicam, phenylbutazone, oxyphenthatrazone, nabumetone, diphenhydramine hydrochloride, clemastine fumarate, brompheniramine maleate, chlorpheniramine maleate, dexchlorpheniramine maleate, triprolidine hydrochloride, promethazine, hydroxyzine hydrochloride, terfenadine, astemizole, azatadine maleate, cyproheptadine hydrochloride, loratadine, carbinoxamine maleate, diphenylpyraline hydrochloride, phenindamine tartrate, tripelennamine hydrochloride, methdilazine hydrochloride, trimprazine tartrate, beclomethasone, dipropionate, triamcinolone acetonide, prednisone, Sumatriptan/Imitrex, Imigran, dihydroergotamine, ergotamine tartrate, dolasetron mesilate, dotarizine, flupirtine, tenosal, tolfenarmic acid, arotinolol, dihydroergocryptine, cyclosporin, azathioprine, methotrexate, glucocorticoids, penicillamine, hydroxychloroquine, colchicine, allopurinol, probenecid, sulfinpyrazone, ginseng, bilberry, black cohosh, cat's claw, cayenne, chamomile, chaste tree, cranberry, echinacea, eleuthero, ephedra, evening primrose oil, feverfew, flax, garlic, ginger, gingko, golden seal, green tea, hawthorn, kava kava, licorice, milk thistle, peppermint, saw palmetto, St. John's wort, methylsulfonyl methane, DMSO, and combinations thereof; and
wherein the composition for transdermal delivery is administered to a human being at least twice during a 24 hour period.
